(19) United States
(12) Patent Application Publication
(10) Pub. No.: US 2024/0376538 A1
(43) Pub. Date: Nov. 14, 2024

(54) PRETREATMENT METHOD FOR DNA ADDUCTOMICS SAMPLES

(71) Applicant: University of South Florida, Tampa, FL (US)

(72) Inventors: Marcus Stanley Cooke, Lutz, FL (US); Carolina Moller, Tampa, FL (US)

(21) Appl. No.: 18/647,858

(22) Filed: Apr. 26, 2024

Related U.S. Application Data
(60) Provisional application No. 63/498,747, filed on Apr. 27, 2023.

(51) Int. Cl. C12Q 1/6872; C12Q 1/6806

(52) U.S. Cl. CPC C12Q 1/6872; C12Q 1/6806; C12Q 2600/154; C12Q 2600/156

(57) ABSTRACT

Disclosed is a method of pretreating a sample including DNA adducts for DNA adductomics, the method including the steps: passing a mobile phase including the sample through a first solid phase extraction (SPE) stationary phase; collecting the mobile phase that passes through the first SPE stationary phase; passing a first eluent through the first SPE stationary phase to elute the DNA adducts; passing the collected mobile phase that passed through the first SPE stationary phase through a second SPE stationary phase; passing a second eluent through the second SPE stationary phase to elute the DNA adducts; combining DNA adducts eluted from the first and second SPE stationary phases; analyzing the DNA adducts by liquid chromatography mass spectrometry (LC-MS); wherein either the first or second SPE stationary phase is a hypercrosslinked, hydroxylated polystyrene-divinylbenzene copolymer and the other SPE stationary phase is a phenyl polymer.

### CROSS REFERENCE TO RELATED APPLICATIONS

This application claims the benefit of priority to U.S. Provisional Applications 63/498,747, filed Apr. 27, 2023, which is incorporated by reference herein in their entireties.

### ACKNOWLEDGEMENT OF GOVERNMENT SUPPORT

This invention was made with government support under grant no. ES030557 awarded by the National Institutes of Health. The government has certain rights in the invention.

### BACKGROUND

The exposome represents the totality of endogenous and exogenous exposures across the lifespan. Exposures may give rise to DNA damage in the form of adducts, which are key factors in the etiology of a variety of human diseases. A DNA adduct can indicate what exposure caused it and the risk of diseases associated with that exposure. Estimates indicate that hundreds of different types of adducts exist in DNA and these numbers increase with environmental exposures. Damaged DNA can block transcription and translation and also lead to mutation, amongst other effects. To prevent this, cells use either direct repair or excision to remove the damaged sections of DNA.

Traditionally DNA damage was directly monitored. Though effective, this method required invasive blood or tissue sampling, DNA isolation, and hydrolysis. Urine is a noninvasive alternative to blood and tissue sampling, but it has its own challenges. Excised DNA adducts are released through urine as a means of disposal. Since these adducts have already been removed from the DNA, there is no need for extensive hydrolysis reactions to isolate the DNA adducts for analysis. Regardless of the method of collection, through urine or blood, DNA adducts are analyzed using liquid chromatography—mass spectrometry (LC-MS).

Analyzing the DNA adducts in urine requires all the extra water, salts and other interfering metabolites to be removed. Failure to remove these will cause matrix effects when analyzing samples on a mass spectrometer, this causes a reduction in signal clarity due to the competing signal from the matrix. The presence of some materials in urine can also damage the LC-MS. This poses a challenge as removing the interfering matrix while retaining a broad range of DNA adducts is no trivial matter. Traditionally this sample preparation is minimized through the use of syringe filters and dilution. However, this may be insufficient to remove the interfering matrix while retaining the all-important DNA adducts.

What are thus needed are new methods of pretreating urine samples, as well as other types of samples, in order to be able to analyze and study the many different DNA adducts (DNA adductomics). The methods disclosed herein address these and other needs.

### SUMMARY

In accordance with the purposes of the disclosed materials and methods, as embodied and broadly described herein, the disclosed subject matter, in one aspect, relates to compounds, compositions, and methods of making and using compounds and compositions. In specific aspects, disclosed is a method of pretreating a sample including DNA adducts for DNA adductomics, the method including the steps: passing a mobile phase including the sample through a first solid phase extraction (SPE) stationary phase; collecting the mobile phase that passes through the first SPE stationary phase; passing a first eluent through the first SPE stationary phase to elute the DNA adducts; passing the collected mobile phase that passed through the first SPE stationary phase through a second SPE stationary phase; passing a second eluent through the second SPE stationary phase to elute the DNA adducts; combining DNA adducts eluted from the first and second SPE stationary phases; analyzing the DNA adducts by liquid chromatography mass spectrometry (LC-MS); wherein either the first or second SPE stationary phase is a hypercrosslinked, hydroxylated polystyrene-divinylbenzene copolymer and the other SPE stationary phase is a phenyl polymer.

Additional advantages will be set forth in part in the following description and in part will be obvious from the description or may be learned by practicing the aspects described below. The advantages described below will be realized and attained by the elements and combinations pointed out in the appended claims. It is to be understood that the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects and together with the description serve to explain the principles of the invention.

FIG. 1 shows method of multiphase SPE extraction, where the flow through from the initial column is retained to go through a secondary column with different physical properties than the first.

FIG. 2 shows combinations of BIOTAGE-ISOLUTE ENV™ with other columns represented as adduct maps, with the control on the left and the standard addition on the right. Key to adduct maps, the color of each point is representative of the signal response from the mass spectrometer.

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, which may, of course, vary. It is also to be understood that the terminology used herein describes particular aspects only and is not intended to be limiting.

Also, throughout this specification, various publications are referenced. The disclosures of these publications in their entireties are hereby incorporated by reference into this application to more fully describe the state of the art to which the disclosed matter pertains. The references disclosed are also individually and expressly incorporated by reference herein for the material contained in them that is discussed in the sentence in which the reference is relied upon.

### General Definitions

In this specification and in the claims that follow, reference will be made to many terms, which shall be defined to have the following meanings:

Throughout the specification and claims, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context dictates otherwise. Thus, for example, reference to “a composition” includes mixtures of two or more such compositions, reference to “an agonist” includes mixtures of two or more such agonists and the like.

“Optional” or “optionally” means that the subsequently described event or circumstance can or cannot occur and that the description includes instances where the event or circumstance occurs and instances where it does not.

The term “analyte” refers to any chemical or biological compound or substance that is subject to the analysis of the disclosure. Analytes can include, but are not limited to, small organic compounds, amino acids, peptides, polypeptides, proteins, nucleic acids, polynucleotides, biomarkers, synthetic or natural polymers, or any combination or mixture thereof.

The term “analyzing” or “analysis” refers to a method by which the quantity of each of the individual analytes described herein is detected. Such analysis may be made using any technique that distinguishes between the analyte and the analyte standard. In some embodiments, the analysis or act of analyzing can include liquid chromatography-mass spectrometry (LC-MS), liquid chromatography-tandem-mass spectrometry (LC-MS-MS).

The term “chromatographic separation” describes a process in which a chemical mixture carried by a liquid or gas is separated into components as a result of differential distribution of the solutes as they flow around or over a stationary liquid or solid phase. For example, chromatographic separations suitable for use in this disclosure can include, but are not limited to solid phase extractions (SPE), liquid chromatographic (including HPLC) methods such as normal-phase HPLC, RP-HPLC, HILIC, and size-exclusion chromatography (SEC), including gel permeation chromatography (GPC). Other suitable methods include additional HPLC methods and related liquid chromatographic techniques, including, e.g., ultra-performance liquid chromatography (UHPLC), fast performance liquid chromatography (FPLC) and the like.

The term “mass spectrometry” is art-recognized to describe an instrumental method for identifying the chemical constitution of a substance by means of the separation of gaseous ions according to their differing mass and charge. A variety of mass spectrometry systems can be employed to analyze the analyte molecules of a sample subjected to the disclosed methods. For example, mass analyzers with high mass accuracy, high sensitivity and high resolution may be used and include, but are not limited to, atmospheric chemical ionization (APCI), chemical ionization (CI), electron impact (EI), fast atom bombardment (FAB), field desorption/field ionization (FD/FI), electrospray ionization (ESI), thermospray ionization (TSP), matrix-assisted laser desorption (MALDI), matrix-assisted laser desorption time-of-flight (MALDI-TOF) mass spectrometers, ESI-TOF mass spectrometers, and Fourier transform ion cyclotron mass analyzers (FT-ICR-MS). In addition, it should be understood that any combination of MS methods could be used in the methods described herein to analyze an analyte in a sample. In certain embodiments, the MS technique used for analysis of the analyte described herein is one that is applicable to most polar compounds, including amino acids, e.g., ESI.

The term “mobile phase” is art-recognized, and describes a solvent system (such as a liquid) used to carry a compound of interest into contact with a solid phase (e.g., a solid phase in a solid phase extraction (SPE) cartridge or HPLC column) and to elute a compound of interest from the solid phase.

The term “sample” refers to a representative portion of a larger whole or group of components that are capable of being separated and detected by the disclosed methods. Exemplary samples include chemically or biologically derived substances, e.g., analytes of the disclosed methods. In particular embodiments, the components of the sample include, but are not limited to small organic compounds, amino acids, peptides, polypeptides, proteins, nucleic acids, polynucleotides, biomarkers, synthetic or natural polymers, or any combination or mixture thereof. The sample can be a body fluid. For example, the sample can be a bodily fluid selected from the group consisting of oral fluids (saliva), sweat, urine, blood, serum, seminal fluid, breast milk, plasma, spinal fluid, and any combination thereof.

### Methods

The continual exposure to exogenous and endogenous risk factors for chronic diseases acts, in part, through the formation of DNA adducts. The untargeted, DNA adductomics approach reflects the totality of expected and unexpected adducts, providing an integrated profile of the types of DNA damage formed. The origin of DNA adducts in urine derives from specific DNA repair processes which yield nucleobase and 2′-deoxyribonucleoside adducts. Therefore, the non-invasive urinary DNA adductomics approach can be used to investigate the precise origin of adducts and their pathway(s) of repair. By LC-HRMS, differences were found in the presence and types of DNA adducts in the urine of DNA repair proficient and deficient mice that were exposed to the toxic agent, benzene.

In some aspects, the techniques described herein relate to a method of pretreating a sample including DNA adducts for DNA adductomics, the method including the steps: passing a mobile phase including the sample through a first solid phase extraction (SPE) stationary phase; collecting the mobile phase that passes through the first SPE stationary phase; passing a first eluent through the first SPE stationary phase to elute the DNA adducts; passing the collected mobile phase that passed through the first SPE stationary phase through a second SPE stationary phase; passing a second eluent through the second SPE stationary phase to elute the DNA adducts; combining DNA adducts eluted from the first and second SPE stationary phases; analyzing the DNA adducts by liquid chromatography mass spectrometry (LC-MS); wherein either the first or second SPE stationary phase is a hypercrosslinked, hydroxylated polystyrene-divinylbenzene copolymer and the other SPE stationary phase is a phenyl polymer.

In some aspects, the first SPE stationary phase is the hypercrosslinked, hydroxylated polystyrene-divinylbenzene copolymer and the second SPE stationary phase is the phenyl polymer. In some aspects, the first SPE stationary phase is the phenyl polymer and the second SPE stationary phase is the hypercrosslinked, hydroxylated polystyrene-divinylbenzene copolymer. The hypercrosslinked, hydroxylated polystyrene-divinylbenzene copolymer can be obtained from commercial sources, e.g., BIOTAGE-ISOLUTE ENV™ from Biotage. Such stationary phases are non-polar, have high capacity, and lack monomers. Such suitable stationary phases also lack C2-C18 alkyl chains and/or silane endcaps. They can have a particle size of from 5 to 500 μm, for example, from 25 to 250, from 50 to 100, from 5 to 100, or from 200 to 500 μm.

Likewise, the phenyl polymer can be 40 to 650 μm in size and can be obtained from commercial sources, e.g., THERMO-HYPERSEP PHE™ from Thermo Fisher. The stationary phases can also be preconditioned before use by, e.g., rinsing with water or a water miscible solvent like methanol, ethanol, propanol, acetone, acetonitrile.

The method can include applying hydrodynamic pressure to cause the sample to flow through the sample channel, across the SPE stationary phases.

In some aspects, the sample is diluted with water before passing through the first SPE stationary phase. Water can be used to reduce viscosity. Further the pH of the sample or mobile phase can be adjusted (made more basic or made more acidic) to better suit binding to the material in the SPE column. In some aspects, the sample is centrifuged before passing through the first SPE stationary phase. The sample is combined with a mobile phase, which can typically include water or water with minor amounts of methanol, ethanol, propanol, acetone, or acetonitrile. When loading the mobile phase with sample onto the stationary phase it can passed through the stationary phase at a flow rate of from 1-7 mL/min, depending on the amount of stationary phase or size.

In some aspects, one or more washes are passed through the first SPE stationary phase before passing the first eluent through, and the one or more washes are combined with the collected mobile phase before passing the collected mobile phase through the second SPE stationary phase. In some aspects, one or more washes are passed through the second SPE stationary phase before passing the second eluent through. The washes can be aqueous media such as water, e.g., with optionally small amounts of methanol, ethanol, formic acid, acetic acid, acetonitrile, or acetone. In some examples, the first and/or second stationary phases are washed once, twice, three, or more times.

In some aspects, the first and/or second eluent is used to elute the DNA adducts from the stationary phases. Suitable examples of the first and/or second eluent are organic solvents such as water, THF, isopropanol, acetonitrile, acetone, or ethyl acetate. The addition of small amounts of organic acid (e.g., acetic acid, formic acid, propionic acid) can be used to improve recoveries.

In some aspects, the DNA adducts are dried and combined with an aqueous solvent before analyzing by LC-MS. Examples of solvents to reconstitute the DNA adducts before analysis are water and methanol.

In some aspects, the mass spectrometry (MS) is tandem MS (MS-MS), matrix-assisted laser desorption/ionization (MALDI) MS, time-of-flight (TOF) MS, or electrospray ionization (ESI) MS.

Other stationary phases that can be used in addition to the first or second stationary phases disclosed herein are BIOTAGE-EVOLUTE EXPRESS ABN™, which is 20 μm styrene copolymer particle having alkylhydroxy groups and 40A pore size, WATERS-OASIS PRIME HLB™, which is a polypropylene reverse phase solid phase extraction media, BIOTAGE-EVOLUTE EXPRESS CX™, or BIOTAGE-EVOLUTE EXPRESS AX™.

### EXAMPLES

The following examples are set forth below to illustrate the methods and results according to the disclosed subject matter. These examples are not intended to include all aspects of the subject matter disclosed herein but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric. Numerous variations and combinations of reaction conditions, e.g., component concentrations, temperatures, pressures, and other reaction ranges and conditions, can be used to optimize the product purity and yield obtained from the described process. Only reasonable and routine experimentation will be required to optimize such process conditions.

Standards used in this series of experiments were individually sourced (Table 2). The SPE used HPLC water (Fisher Chemical) and methanol (Fisher Chemical), samples were combined with 50 mM ammonium acetate (Sigma Aldrich). Additionally, the HPLC solvent gradient the aqueous phase consisted of HPLC water spiked with 10 mM ammonium acetate, the organic phase was 95% HPLC methanol with a 0.1% formic acid (Fisher Chemical) spike.

### Example 1

The study used male and female C57BL6/J mice (n=8), aged around 12-13 weeks, obtained from Jackson Laboratories (Strain #:000664) and housed in a certified facility compliant with the standards of the Association for Assessment and Accreditation of Laboratory Animal Care International (AALAC). Their care and management adhered to guidelines including the “Guide for the Care and Use of Laboratory Animals,” Animal Welfare Regulations Title 9 Code of Federal Regulations Subchapter A, “Animal Welfare” (Parts 1-3), the Public Health Service Policy on Humane Care and Use of Laboratory Animals (PHS Policy), and the University of South Florida Institutional Animal Care and Use Committee's Principles and Procedures of Animal Care and Use (IACUC Principles). All mice were acclimated for a minimum of two weeks before use. For urine collections, mice were randomly assigned and temporarily housed in metabolic cages aligning with ethical and procedural standards for animal care and research practices. A maximum of three mice were housed per cage to collect daily urine and feces samples after 24 h period. All urine was pooled and aliquoted upon collection and stored at −80° C. until used.

Human urine was commercially obtained from Innovative Research. As a method of cleaning and homogenizing, each urine aliquot was centrifuged and only the supernatant was collected for analysis.

### Pre-Treatment Procedure for Urinary Extraction of DNA Adducts by Manual SPE

To determine the SPE column with the best ability for isolating the DNA adducts, a series of six columns each with different stationary phases and chemical properties was selected (Table 1). The efficiency of the columns was determined individually before the columns were combined in a sequential SPE method to determine which combination of columns worked best. Each column uses 250 μL of urine that has been diluted by half with HPLC grade water. Samples were run in triplicate for the method development, 750 μL of urine is required for each trial.

TABLE 1
SPE cartridges used to determine their efficiency in
isolating DNA adducts from urine samples.

| | | Main retention |
|---|---|---|
| Column | Stationary phase | mechanism |
| PHE | Phenyl | Pi-Pi interactions |
| ENV | hyper crosslinked | non-polar SPE phase |
| | hydroxylated | |
| | polystyrene- | |
| | divinylbenzene | |
| | copolymer | |
| ABN | modified PS-DVB | polar (hydrophilic) |
| | polymeric | and non-polar |
| | | (hydrophobic) |
| | | interactions |
| HLB | Supra-Poly | extract mid-polar and |
| | Hydrophilic/ | non-polar compounds |
| | Lipophilic Balanced | |
| CX | sulfonic acid groups | Reverse phase, cation |
| | | exchange |
| AX | quaternary amine | Reverse phase, anion |
| | groups | exchange |

In order to test the efficiency of each column in isolating the DNA adducts from a urine matrix, samples were spiked with a cocktail of 20 standards each with a final concentration of 1 μg/mL (Table 2). By selecting a wide variety of adducts and using targeted analysis the exact recovery efficiency of each SPE column can be determined. To achieve this, two samples of urine were run in tandem. To obtain the % recovery both spiked before and after are needed. By comparing the eluent from the SPE cartridges the recovery of each individual column and each combination of columns was determined.

TABLE 2
Standards used in SPE analysis. Initially a limited cocktail
of the following DNA adducts was used to determine the
efficiency of each column. To ensure consistency, 50 μL of
a standard concentration of 1 mg/mL of each standard was
spiked into the urine samples. The complete list of
standards was used in secondary testing of columns.

| | | M/Z | M/Z |
|---|---|---|---|
| Standard | RT | [M + H]⁺ | [M + H-dR]⁺ |
| 6-Methylaminopurine | 16.977 | 150.0772 | N/A |
| 5-Methylcytosine | 5.866 | 126.0661 | N/A |
| 7-methylguanine | 11.906 | 166.0726 | N/A |
| 5-carboxycytosine | 2.113 | 156.0411 | N/A |
| Isoguanine | 5.077 | 152.0565 | N/A |
| Cytosine | 7.815 | 112.0503 | N/A |
| Adenine | 10.779 | 136.0618 | N/A |
| Guanine | 6.260 | 152.0559 | N/A |
| Thymine | 9.032 | 127.0502 | N/A |
| 5-(Hydroxymethyl)cytosine | 4.142 | 142.0858 | N/A |
| 6-O-Methylguanine | 17.721 | 166.0722 | N/A |
| 9-Methyladenine | 11.850 | 149.0453 | N/A |
| 1,N6-Ethenoadenine | 17.597 | 160.0617 | N/A |
| 9-ethylguanine | 17.901 | 180.0880 | N/A |
| 2′-deoxycytidine | 7.804 | 228.0974 | 112.0503 |
| N-Benzyl-2′-deoxycytidine | 40.698 | 332.2415 | 216.0759 |
| 2′-deoxyuridine | 5.933 | 251.0626 | 113.0341 |
| 2′-deoxyadenosine | 18.881 | 252.1088 | 136.0615 |
| 2′-deoxyguanosine | 13.551 | 268.1040 | 152.0563 |
| 5-ethyl-2′-deoxyuridine | 12.130 | 275.0623 | 159.0157 |
| 8-oxo-2′-deoxyguanosine | 18.205 | 284.0997 | 168.1167 |
| 2-O-ethylthymidine | 25.068 | 271.1282 | 155.0819 |
| Adenosine | 17.766 | 268.1040 | 136.0617 |
| Guanosine | 18.205 | 284.0997 | 152.0566 |
| N4-Anizoyl-2′-deoxycytidine | 45.723 | 362.1570 | 246.1479 |
| N3-Methylthymidine | 23.592 | 257.1105 | 141.0657 |
| 8-oxo-2′-deoxyadenosine | 20.966 | 268.1046 | 152.0563 |
| 8-oxoadenosine | 15.726 | 284.0953 | 168.0510 |
| 2′-deoxy-N-ethylguanosine | 26.476 | 296.1353 | 180.0881 |
| O6-(2-hydroxyethyl)-2′- | 23.727 | 312.1290 | 196.0832 |
| deoxyguanosine | | | |
| 1,N6-Etheno-2′-deoxyadenosine | 22.792 | 276.1094 | 160.0618 |
| N6-methyl-2′-deoxyadenosine | 26.161 | 266.1253 | 150.0772 |
| 5-ethynyl-2′-deoxycytidine | 16.368 | 252.0958 | 136.0502 |
| 5-methyl-2′-deoxycytidine | 12.176 | 242.1131 | 126.0662 |
| 5-ethynyl-2′-deoxyuridine | 12.130 | 253.0807 | 137.0339 |

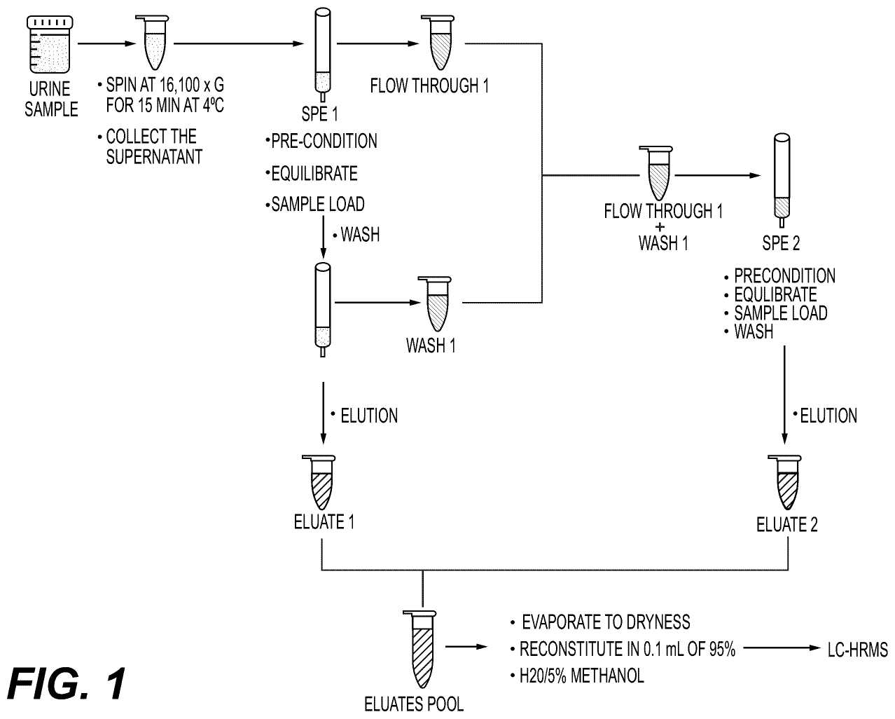

Initially, to establish their extraction efficiency, each type of SPE column was evaluated separately. Then, each combination of two columns was assessed performing two rounds of SPE as follows: Urine was centrifuged at 16,100×g for 15 min and the collected supernatant was aliquoted into three vials with labels identifying them as diluted urine, spiked urine before SPE, and spiked urine after SPE. A stock solution of a mixture of the 20 standards containing unmodified, modified nucleobases and 2′-deoxynucleosides was prepared for a final concentration of 10 mg/mL each. This stock solution was used for spiking the urine sample for a resulting concentration of 1 mg/mL for each standard. For the other samples, diluted urine and spiked urine after SPE, the corresponding volume of HPLC-grade water was added up to the same final volume. Because different cartridges with different sorbents were used, each extraction was performed following the manufacturer's protocol. In general, each extraction complied with the SPE steps of the preconditioning, sample loading, washing and elution process (FIG. 1). After the precondition, 500 mL of each urine sample was combined with 500 mL of 50 mM of ammonium acetate, pH 6. For the first extraction (SPE-1), samples were loaded into the respective cartridges and the flow-throughs were collected. Additionally, all the solutions collected during the wash step were saved. After the elution step, the eluate of the SPE-1 was saved. Both the collected flow-through and the wash from the SPE-1 were combined and further extracted using a second type of SPE cartridge with a different sorbent (SPE-2). After following the manufacturing recommendation steps for the SPE-2, the second eluate was combined with the first one. All the samples were evaporated to dryness and reconstituted in 100 mL of 95% Water, 5% Methanol, and centrifuged at 5000×g for 5 min. The supernatants were aliquoted in vials and then proceeded to the LC-MS analysis (FIG. 1).

### Liquid Chromatography Mass Spectrometry

The LC Q-TOF MS methods were optimized to maximize the analytical performance for urinary DNA adductomic analysis. Some of these parameters were already described in the literature. The method was developed using a LC Q-TOF MS 6540 (Agilent Technologies, Santa Clara, CA, USA). This instrument utilizes jet streaming electrospray ionization (ESI) set to positive mode polarity. The column utilized was an Inertsil ODS-3 C18 column (150×2.1 mm i.d., 5 μm) from GL Sciences (Tokyo, Japan). The mobile phase comprised 1 mM aqueous ammonium acetate solution as mobile phase A, and 95% methanol (v/v) containing 0.1% (v/v) formic acid as mobile phase B. The gradient program established consisted in 0.5% of mobile phase B at 0-2 min, 30% of mobile phase B at 32 min, and 99.5% of mobile phase B at 48 min with a 3 min hold, and then a 9 min re-equilibration to the initial proportion (Table 3). The column temperature was set to 40° C. The flow rate was 0.2 mL/min and the total run time was 60 min. The injection volume was 20 μL. The analyses were performed using full scan mode. For MS1 the mass range was restricted to m/z 50-600. The source parameters determined were: gas temperature of 325° C. with a gas flow of 8 L/min, nebulizer at 35 psig, and sheath gas temperature of 400° C. with a flow of 12 L/min. The scan source parameters determined were: a VCap of 3500 with fragmentor voltage set to 125 V. For MS2, the acquisition was achieved in auto MS/MS with collision energies of 10, 20, 40, and 60 mV. The software used for data processing and chromatogram peak identification was the Agilent (Santa Clara, CA) MassHunter Qualitative Analysis version 7.0. Peaks corresponding to each analyte were individually and manually analyzed. The identification of the standards was achieved taking into account the accurate mass of precursor and neutral loss of 116 amu corresponding to the loss of the 2′-deoxyribose group (dR).

TABLE 3
Gradient used for elution of analytes from an
Intersil ODS −3 reverse phase column.

| Gradient | Percent 1 mM | Percent 95% MeOH |
|---|---|---|
| change times | ammonium acetate | 0.1% formic acid |
| Start: 0 min | 99.5% | 0.5% |
| 2 min | 99.5% | 0.5% |
| 32 min | 70% | 30% |
| 48 min | 0.5% | 99.5% |
| 51 min | 99.5% | 0.5% |
| End: 60 min | 99.5% | 0.5% |

As confirmation, an alternat instrument, THERMO VANQUISH NEO UHPLC—THERMO Q-EXACTIVE PLUS™, was used to confirm the analysis of the first. Data was analyzed with the Feature Hunter 1.3 software. This allowed confirmation of the presence of the adducts in the SPE eluate.

Initially columns were used individually to determine the ability of the individual column to retain the analytes of interest. The extraction efficiency of each type of SPE and their combination was achieved by the recovery of the standards using the peak intensity/abundance detected in MS¹. All the analytes detected were identified by the theorical mass of the precursor and the mass product ions obtained by MS². The percent recovery was calculated by comparing a urine sample that had the standards spiked in before the SPE to a sample that had the standards spiked in after the SPE. By monitoring the ability of the individual column, we predicted that a combination including BIOTAGE-ISOLUTE ENV™ would yield the best results. This is due to the 16 retained analytes out of the 20 initially injected, compared to the column BIOTAGE-EVOLUTE EXPRESS CX™ which also retained 14 of the analytes (Table 4).

TABLE 4
Individual column percent recovery of spiked in analytes. The highlighted
sections indicate an analyte that had more than 80% recovery.

| | BIOTAGE- | | WATERS- | BIOTAGE- | BIOTAGE- | |
|---|---|---|---|---|---|---|
| | EVOLUTE | BIOTAGE- | OASIS | EVOLUTE | EVOLUTE | THERMO- |
| | EXPRESS | ISOLUTE | PRIME | EXPRESS | EXPRESS | HYPERSEP |
| Analyte | ABN ™ | ENV ™ | HLB ™ | CX ™ | AX ™ | PHE ™ |
| 6-Methylaminopurine | 107% | 94% | 117% | 93% | 96% | 106% |
| 5-Methylcytosine | 16% | 77% | 22% | 112% | 31% | 25% |
| 7-methylguanine | 52% | 91% | 60% | 115% | 84% | 108% |
| 5-carboxycytosine | 0% | 0% | 2% | 49% | 49% | 179% |
| Isoguanine | 7% | 72% | 0% | 151% | 63% | 59% |
| Cytosine | 20% | 37% | 4% | 0% | 32% | 12% |
| Adenine | 24% | 124% | 24% | 133% | 94% | 94% |
| Guanine | 124% | 75% | 73% | 94% | 195% | 19% |
| Thymine | 0% | 100% | 7% | 0% | 100% | 11% |
| 3-methyl-adenine | 74% | 95% | 103% | 141% | 69% | 109% |
| 2′-deoxycytidine | 45% | 124% | 32% | 97% | 44% | 13% |
| N-Benzyl-2′- | 166% | 57% | 136% | 122% | 108% | 114% |
| deoxycytidine | | | | | | |
| 2′-deoxyuridine | 26% | 57% | 14% | 0% | 26% | 139% |
| 2′-deoxyadenosine | 127% | 95% | 122% | 75% | 41% | 80% |
| 2′-deoxyguanosine | 43% | 101% | 49% | 96% | 84% | 0% |
| 5-ethyl-2′-deoxyuridine | 20% | 189% | 27% | 0% | 30% | Not |
| | | | | | | Recovered |
| 8-oxo-2′-deoxyguanosine | 0% | 96% | 0% | Not | 18% | 74% |
| | | | | Recovered | | |
| 2-O-ethylthymidine | 97% | 75% | 110% | 93% | 74% | 87% |
| Adenosine | 63% | 118% | 117% | 94% | 23% | 30% |
| Guanosine | 0% | 107% | 0% | 75% | 38% | 65% |

### Combined SPE Column Analyte Recovery

BIOTAGE-ISOLUTE ENV™, an example of a hypercrosslinked, hydroxylated polystyrene-divinylbenzene copolymer, was selected as the base for further extractions, due to initial individual column recovery. BIOTAGE-ISOLUTE ENV™ was combined with each of the other SPE columns in order to improve the retention of the cocktail of standards. Initially single trial of each combination was done, though BIOTAGE-ISOLUTE ENV™ to BIOTAGE-EVOLUTE EXPRESS CX™ showed promise for a comprehensive recovery having to change the pH of the solvents to elute the adducts from the column has the potential to destabilize and degrade DNA. For this reason, both columns BIOTAGE-EVOLUTE EXPRESS CX™ and BIOTAGE-EVOLUTE EXPRESS AX™ were retired from the selection process.

The remaining columns were run in combination with BIOTAGE-ISOLUTE ENV™ in triplicate over the course of several months. This allowed the determination of whether the recovery of the column is stable, in Table 5 the standards that are green or gold filled showed an average recovery above 80%. However, when the variation between trials is taken into consideration only those standards that are highlighted Green have an average and deviation that remains above 80% recovery. For this reason, the column combination BIOTAGE-ISOLUTE ENV™ to THERMO-HYPERSEP PHE™ was chosen as the optimal combination, with 75% of the spiked in adducts being successfully recovered across multiple trials. Compare this to BIOTAGE-ISOLUTE ENV™ to BIOTAGE-EVOLUTE EXPRESS ABN™ combination at 50% or BIOTAGE-ISOLUTE ENV™ to WATERS-OASIS PRIME HLB™ at 40% of the adducts spiked in being recovered.

TABLE 5
BIOTAGE-ISOLUTE ENV ™ combined with other columns recovery.

| | BIOTAGE- ISOLUTE ENV ™ | | | | |
|---|---|---|---|---|---|
| | Combined with | | | | |
| | BIOTAGE- | WATERS- | | BIOTAGE- | BIOTAGE- |
| | EVOLUTE | OASIS | THERMO- | EVOLUTE | EVOLUTE |
| | EXPRESS | PRIME | HYPERSEP | EXPRESS | EXPRESS |
| Analyte | ABN ™ | HLB ™ | PHE ™ | CX ™ | AX ™ |
| 6-Methylaminopurine | 102% ± 18% | 120% ± 15% | 99% ± 14% | 122% | 59% |
| 5-Methylcytosine | 91% ± 25% | 147% ± 50% | 94% ± 8% | 114% | 65% |
| 7-methylguanine | 118% ± 84% | 252% ± 157% | 98% ± 12% | 120% | 108% |
| 5-carboxycytosine | 160% ± 106% | 182% ± 132% | 41% ± 43% | 38% | 21% |
| Isoguanine | 131% ± 22% | 234% ± 87% | 97% ± 27% | 129% | 37% |
| Cytosine | 106% ± 38% | 140% ± 83% | 64% ± 49% | 115% | 7% |
| Adenine | 129% ± 12% | 157% ± 94% | 100% ± 11% | 101% | 92% |
| Guanine | 115% ± 14% | 308% ± 260% | 141% ± 55% | 82% | 42% |
| Thymine | 135% ± 21% | 187% ± 155% | 100% ± 11% | 95% | 91% |
| 3-methyl-adenine | 106% ± 17% | 140% ± 34% | 84% ± 7% | 92% | 54% |
| 2′-deoxycytidine | 117% ± 8% | 111% ± 41% | 148% ± 63% | 77% | 47% |
| N-Benzyl-2′- | 101% ± 5% | 149% ± 21% | 92% ± 9% | 134% | 152% |
| deoxycytidine | | | | | |
| 2′-deoxyuridine | Not | Not | 92% ± 23% | 131% | 37% |
| | recovered | recovered | | | |
| 2′-deoxyadenosine | 100% ± 13% | 196% ± 94% | 149% ± 62% | 119% | 57% |
| 2′-deoxyguanosine | 88% ± 27% | Not | 132% ± 51% | 101% | 117% |
| | | recovered | | | |
| 5-ethyl-2′- | Not | Not | 113% ± 5% | 115% | 49% |
| deoxyuridine | recovered | recovered | | | |
| 8-oxo-2′- | 136% ± 53% | Not | 82% ± 42% | 106% | 71% |
| deoxyguanosine | | recovered | | | |
| 2-O-ethylthymidine | 97% ± 12% | 135% ± 28% | 107% ± 7% | 96% | 98% |
| Adenosine | 74% ± 25% | 136% ± 89% | 100% ± 0% | 136% | 68% |
| Guanosine | 290% ± 218% | 216% ± 238% | 106% ± 27% | 126% | 82% |

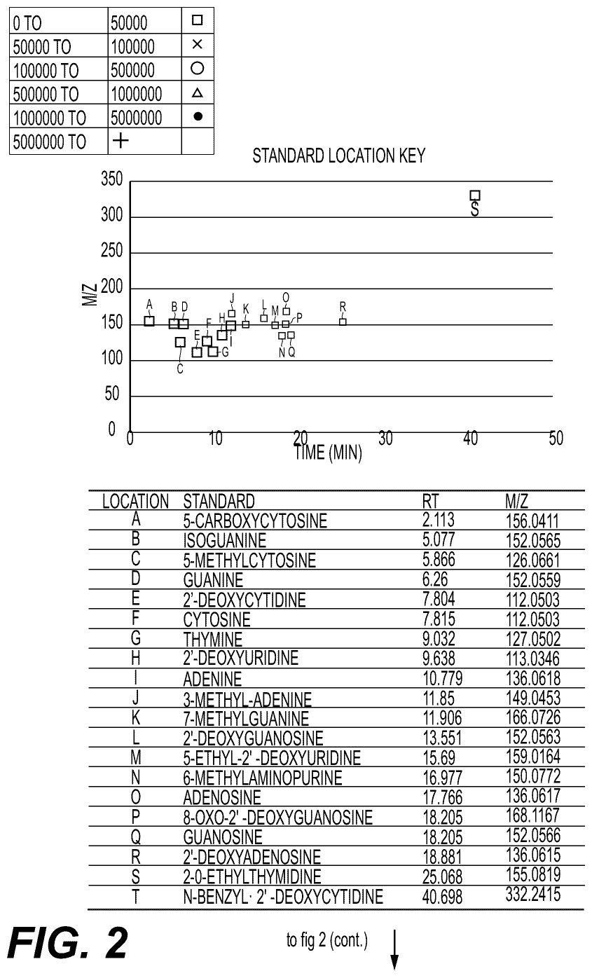

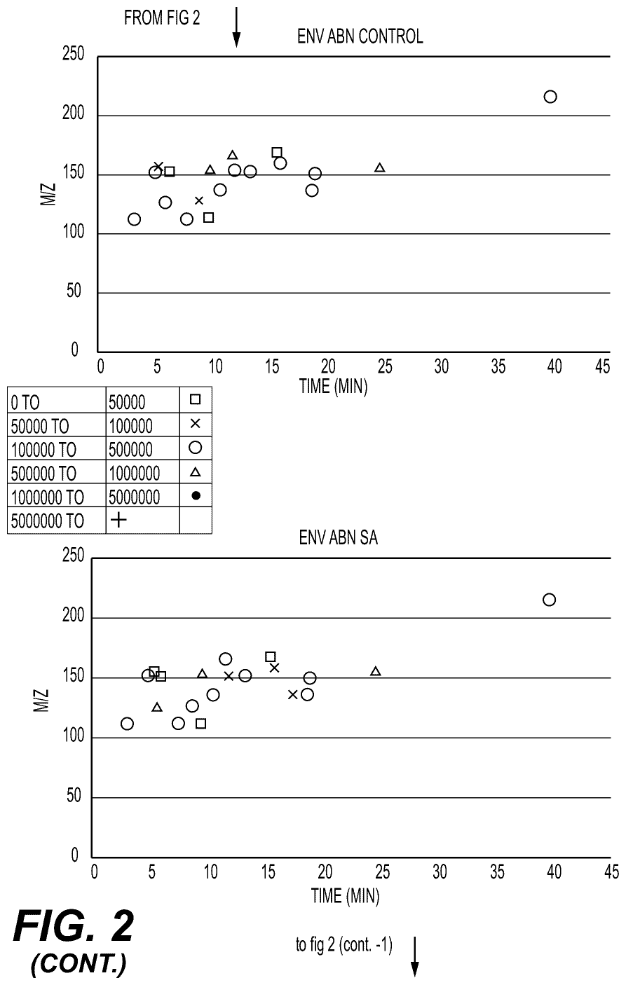

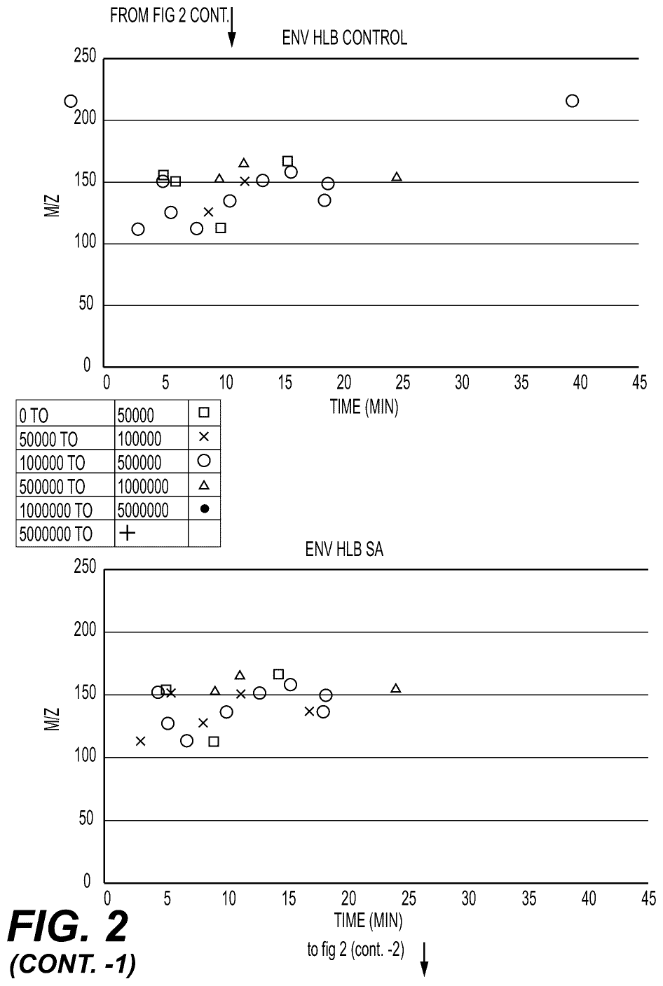

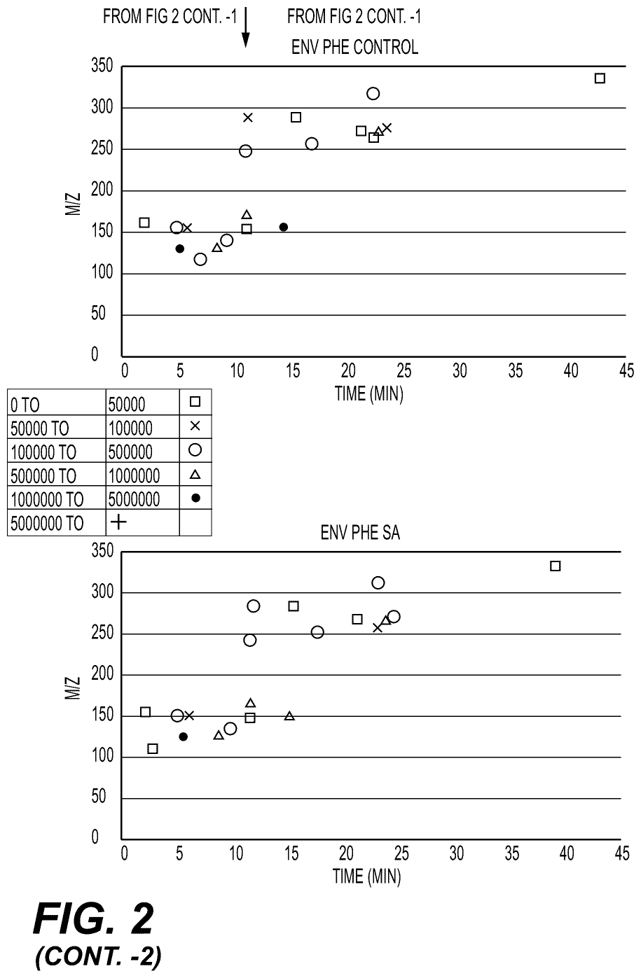

An alternative method of visualizing the recovery of the column combinations is to use adduct maps. These maps display both the retention time (x axis) and the mass (y axis). The color of each point indicates the signal intensity of the individual analyte (FIG. 2). The maps that have been labeled control indicate that the standards were added to the urine after the SPE, these represent what the instrument response would be if there was complete efficiency in retaining the analytes. The maps that have been labeled SA (standard addition), represent the samples where the analytes were added to the urine before the SPE. These represent the actual ability of the column to retain the analytes. By comparing the two maps a visual representation of the retention of each adduct can be established.

Adduct maps give a visual representation of the recovery of each adduct. By comparing the control, which represents a complete recovery of the adducts from the SPE, to the standard addition (SA) maps, which represented the true column recovery, we can draw a visual representation of the data presented in Table 5.

### Alternate Order SPE Column Analyte Recovery

In addition to which column combination allowed for the best retention of analytes, we determined that the order of the columns also matters. Having established that BIOTAGE-ISOLUTE ENV™+THERMO-HYPERSEP PHE™ combination was the most efficient, the order was swapped to THERMO-HYPERSEP PHE™+BIOTAGE-ISOLUTE ENV™ Though the columns were treated the same and in theory the order should not be significant, it was found that by using the column with the higher initial efficiency first the recovery was improved. In this instance BIOTAGE-ISOLUTE ENV™ had a higher individual recovery of analytes than THERMO-HYPERSEP PHE™, 16 out of 20 rather than 9 of 20 (Table 4). When the more efficient column was run first (BIOTAGE-ISOLUTE ENV™+THERMO-HYPERSEP PHE™) there was complete recovery of samples, whereas the reversed (THERMO-HYPERSEP PHE™+BIOTAGE-ISOLUTE ENV™) lost 7 samples during the SPE (Table 6).

TABLE 6
BIOTAGE-ISOLUTE ENV ™
THERMO-HYPERSEP PHE ™ swap.

| | BIOTAGE- | THERMO- |
|---|---|---|
| | ISOLUTE | HYPERSEP |
| | ENV ™ + | PHE ™ + |
| | THERMO- | BIOTAGE- |
| | HYPERSEP | ISOLUTE |
| Analyte | PHE ™ | ENV ™ |
| 6-Methylaminopurine | 113% ± 4% | 75% ± 12% |
| 5-Methylcytosine | 132% ± 36% | 93% ± 7% |
| 7-methylguanine | 96% ± 19% | 93% ± 11% |
| 5-carboxycytosine | 94% ± 60% | 161% ± 100% |
| Isoguanine | 130% ± 62% | 118% ± 45% |
| Cytosine | 92% ± 23% | 28% ± 40% |
| Adenine | 93% ± 16% | 86% ± 26% |
| Guanine | 146% ± 15% | 61% ± 48% |
| Thymine | 85% ± 10% | 67% ± 42% |
| 5-(Hydroxy- | 82% ± 38% | 135% ± 81% |
| methyl)cytosine | | |
| 6-O-Methylguanine | 109% ± 19% | 161% ± 52% |
| 9-Methyladenine | 111% ± 25% | 130% ± 64% |
| 1,N6-Ethenoadenine | 158% ± 50% | 128% ± 97% |
| 9-ethylguanine | 119% ± 12% | 117% ± 29% |
| 2′-deoxycytidine | 96% ± 12% | 87% ± 20% |
| 2′-deoxyadenosine | 150% ± 49% | 76% ± 19% |
| 2′-deoxyguanosine | 131% ± 18% | 89% ± 5% |
| 2-O-ethylthymidine | 113% ± 15% | 113% ± 10% |
| N4-Anizoyl-2′- | 115% ± 13% | 336% ± 187% |
| deoxycytidine | | |
| N3-Methylthymidine | 101% ± 12% | 105% ± 16% |
| 2′-deoxy-N- | 104% ± 6% | 130% ± 40% |
| ethylguanosine | | |
| O6-(2-hydroxyethyl)-2′- | 116% ± 15% | 107% ± 25% |
| deoxyguanosine | | |
| 1,N6-Etheno-2′- | 110% ± 17% | 102% ± 4% |
| deoxyadenosine | | |
| N6-methyl-2′- | 111% ± 11% | 75% ± 16% |
| deoxyadenosine | | |
| 5-ethynyl-2′- | 115% ± 0% | 90% ± 7% |
| deoxycytidine | | |
| 5-methyl-2′- | 140% ± 48% | 70% ± 21% |
| deoxycytidine | | |
| N-Benzyl-2′- | 744% ± 876% | Not Recovered |
| deoxycytidine | | |
| 2′-deoxyuridine | Not Recovered | Not Recovered |
| 5-ethyl-2′-deoxyuridine | Not Recovered | Not Recovered |
| 8-oxo-2′-deoxyguanosine | Not Recovered | Not Recovered |
| Adenosine | Not Recovered | Not Recovered |
| Guanosine | Not Recovered | 105% ± 21% |
| 8-oxo-2′-deoxyadenosine | Not Recovered | Not Recovered |
| 8-oxoadenosine | Not Recovered | Not Recovered |
| 5-ethynyl-2′- | Not Recovered | Not Recovered |
| deoxyuridine | | |

The combination of the BIOTAGE-ISOLUTE ENV™+THERMO-HYPERSEP PHE™ cartridges provided the best analyte recoveries. Initial results show that leveraging and combining the different characteristics of SPE sorbents decreases the matrix interference without compromising the recovery of the DNA adducts, providing a highly effective clean-up prior to urinary DNA adductomics.

### Example 2

DNA repair proficient and deficient mice were exposed to benzene (500 mg/kg, i.p.). The mice were placed in metabolic cages for the collection of daily urine samples over 21 days. To decrease the matrix effects, a combination of two SPE cartridges with different sorbents was used. The urine samples were first passed through the BIOTAGE-ISOLUTE ENV™+SPE cartridge and the collected flow-through and wash were subjected to an SPE via a second, Hypersep-Phenyl cartridge. The eluates of both cartridges were combined prior to analysis by LC-QTOF-MS/MS² operated in positive ESI mode. The MS software, MassHunter (Agilent) and MS Workbook-Suite Enterprise (ACD/Labs were used for data analysis including features such as neutral loss of 2-deoxyribose, specific product ions, and adductomic maps.

The combination of two SPE pretreatment decrease the urine matrix interference without compromising the recovery of the DNA adducts, providing an effective urinary DNA adductomic analysis. By using human and mice urine spiked with a mixture of DNA adducts standards, and a specific combination of two different SPE sorbents BIOTAGE-ISOLUTE ENV™ followed by THERMO-HYPERSEP PHE™, provided better extraction recoveries, and for the widest range of the adduct standards.

The optimized LC-QTOF-MS¹/MS² method following the SPE pretreatment was successfully applied in the non-targeted detection of benzene-induced DNA adducts in mouse urine. First, the presence of benzene metabolites in the urine was established in order to define the relationship between exposure and the associated DNA adductome in mice. No benzene metabolites were detected in the control group. As expected, benzene metabolites were detected in the benzene-treated groups. Mouse urines were further analyzed to determine the presence of DNA adducts following the benzene exposure. Variations in the profile of adducts present in urine were noted, such as the presence/absence of the ion peaks, together with changes in ion peak intensity. This was attributed to differences in the DNA repair pathways available between the repair proficient and deficient mice. Additionally, novel DNA adducts originating from the specific in vivo metabolism of benzene were detected in all the treatment groups.

What is claimed is:

1. A method of pretreating a sample comprising DNA adducts for DNA adductomics, the method comprising:
passing a mobile phase comprising the sample through a first solid phase extraction (SPE) stationary phase;
collecting the mobile phase that passes through the first SPE stationary phase;
passing a first eluent through the first SPE stationary phase to elute the DNA adducts;
passing the collected mobile phase that passed through the first SPE stationary phase through a second SPE stationary phase;
passing a second eluent through the second SPE stationary phase to elute the DNA adducts;
combining DNA adducts eluted from the first and second SPE stationary phases; and
analyzing the DNA adducts by liquid chromatography mass spectrometry (LC-MS);
wherein either the first or second SPE stationary phase is a hypercrosslinked, hydroxylated polystyrene-divinylbenzene copolymer and the other SPE stationary phase is a phenyl polymer.

2. The method of claim 1, wherein the first SPE stationary phase is the hypercrosslinked, hydroxylated polystyrene-divinylbenzene copolymer and the second SPE stationary phase is the phenyl polymer.

3. The method of claim 1, wherein the first SPE stationary phase is the phenyl polymer and the second SPE stationary phase is the hypercrosslinked, hydroxylated polystyrene-divinylbenzene copolymer.

4. The method of claim 1, wherein the sample is saliva, sweat, urine, blood, serum, seminal fluid, breast milk, plasma, spinal fluid, or any combination thereof.

5. The method of claim 1, wherein the sample is urine.

6. The method of claim 1, wherein the sample is diluted with water before passing through the first SPE stationary phase.

7. The method of claim 1, wherein the sample is centrifuged before passing through the first SPE stationary phase.

8. The method of claim 1, wherein one or more washes are passed through the first SPE stationary phase before passing the first eluent through, and the one or more washes are combined with the collected mobile phase before passing the collected mobile phase through the second SPE stationary phase.

9. The method of claim 1, wherein one or more washes are passed through the second SPE stationary phase before passing the second eluent through.

10. The method of claim 1, wherein the DNA adducts are dried and combined with an aqueous solvent before analyzing by LC-MS.

11. The method of claim 10, wherein the solvent comprises water and methanol.

12. The method of claim 1, wherein the mass spectrometry (MS) is tandem MS (MS-MS), matrix-assisted laser desorption/ionization (MALDI) MS, time-of-flight (TOF) MS, or electrospray ionization (ESI) MS.
